(19) United States
(12) Patent Application Publication
(10) Pub. No.: US 2022/0280760 A1
(43) Pub. Date: Sep. 8, 2022

(54) MECHANICALLY EXPANDABLE SHUNT IMPLANT

(71) Applicant: Edwards Lifesciences Corporation, Irvine, CA (US)

(72) Inventors: Linda Thai, Mission Viejo, CA (US); Zachary Charles VanEvery, Irvine, CA (US); Tarannum Ishaq Gutierrez, Ladera Ranch, CA (US); Denis Tauz, Laguna Hills, CA (US); Cooper Ryan Rickerson, Huntington Beach, CA (US)

(21) Appl. No.: 17/750,236

(22) Filed: May 20, 2022

Related U.S. Application Data
(63) Continuation of application No. PCT/US2020/058784, filed on Nov. 4, 2020.
(60) Provisional application No. 62/939,407, filed on Nov. 22, 2019.

(51) Int. Cl. A61M 27/00

(52) U.S. Cl. CPC A61M 27/002; A61M 2205/0266

(57) ABSTRACT

A shunt comprises a central flow portion configured to fit at least partially within an opening in a tissue wall. The tissue wall is situated between a first anatomical chamber and a second anatomical chamber and the opening represents a blood flow path between the first anatomical chamber to the second anatomical chamber. The central flow portion is further configured to maintain the blood flow path from the first anatomical chamber to the second anatomical chamber, prevent in-growth of tissue within the opening, and expand in response to expansion of the tissue wall.

### RELATED APPLICATIONS

This application is a continuation of International Patent Application No. PCT/US2020/058784, filed on Nov. 4, 2020, entitled MECHANICALLY EXPANDABLE SHUNT IMPLANT, which claims priority to U.S. Provisional Application No. 62/939,407, filed on Nov. 22, 2019, entitled MECHANICALLY EXPANDABLE SHUNT IMPLANT. Each of these disclosures is hereby incorporated by reference in its entirety.

### BACKGROUND

The present invention relates generally to cardiac shunts and systems and methods of delivery, and in particular, to a shunt to reduce left atrial pressure.

Heart failure is a common and potentially lethal condition affecting humans, with sub-optimal clinical outcomes often resulting in symptoms, morbidity and/or mortality, despite maximal medical treatment. In particular, “diastolic heart failure” refers to the clinical syndrome of heart failure occurring in the context of preserved left ventricular systolic function (ejection fraction) and in the absence of major valvular disease. This condition is characterized by a stiff left ventricle with decreased compliance and impaired relaxation, which leads to increased end-diastolic pressure. Approximately one third of patients with heart failure have diastolic heart failure and there are very few, if any, proven effective treatments.

Symptoms of diastolic heart failure are due, at least in a large part, to an elevation in pressure in the left atrium. Elevated Left Atrial Pressure (LAP) is present in several abnormal heart conditions, including Heart Failure (HF). In addition to diastolic heart failure, a number of other medical conditions, including systolic dysfunction of the left ventricle and valve disease, can lead to elevated pressures in the left atrium. Both Heart Failure with Preserved Ejection Fraction (HFpEF) and Heart Failure with Reduced Ejection Fraction (HFrEF) can exhibit elevated LAP. It has been hypothesized that both subgroups of HF might benefit from a reduction in LAP, which in turn reduces the systolic preload on the left ventricle, Left Ventricular End Diastolic Pressure (LVEDP). It could also relieve pressure on the pulmonary circulation, reducing the risk of pulmonary edema, improving respiration and improving patient comfort.

### SUMMARY

For purposes of summarizing the disclosure, certain aspects, advantages and novel features have been described herein. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

Some implementations of the present disclosure relate to a shunt comprising a central flow portion configured to fit at least partially within an opening in a tissue wall. The tissue wall is situated between a first anatomical chamber and a second anatomical chamber and the opening represents a blood flow path between the first anatomical chamber to the second anatomical chamber, The central flow portion is further configured to maintain the blood flow path from the first anatomical chamber to the second anatomical chamber, prevent in-growth of tissue within the opening, and expand in response to expansion of the tissue wall.

The shunt may further comprise one or more anchoring arms, which may also be referred to as “means for anchoring,” extending from the central flow portion. The one or more anchoring arms may be configured to anchor to the tissue wall. In some embodiments, each of the one or more anchoring arms may include an anchoring mechanism at an end portion. The anchoring mechanism may comprise one or more of a group including a barb, a hook, a nail, and a screw.

In some embodiments, the central flow portion comprises a network of one or more lines and each of the one or more lines is configured to interweave with itself or at least one other line of the one or more lines. The central flow portion may comprise a network of chains and each chain of the network of chains may be configured to interlock with at least one other chain of the network of chains.

The central flow portion may comprise a coiled line. In some embodiments, the central flow portion has a fixed diameter approximately equal to a diameter of the opening. A first portion of the central flow portion may be configured to be situated within the opening and a second portion of the central flow portion may be configured to extend into the first anatomical chamber. The first portion may have a first diameter and the second portion may have a second diameter. The second diameter may be greater than the first diameter and/or the first diameter may be lesser than the second diameter. The second portion may be configured to prevent dislodging of the central flow portion.

In some embodiments, the central flow portion comprises one or more rings. Each of the one or more rings may have an elliptical shape to approximate a shape of the opening. In some embodiments, at least one of the one or more rings is coated in a polymer configured to prevent tissue growth. Each of the one or more rings may be composed of a shape-memory material. In some embodiments, each of the one or more rings may be configured to naturally assume a first diameter. Each of the one or more rings may be configured to be compressed to a second diameter that is smaller than the first diameter to fit into the opening. In some embodiments, each of the one or more rings is configured to press against the tissue wall to hold itself in place. Each of the one or more rings may comprise an anchoring mechanism configured to anchor to the tissue wall. In some embodiments, the anchoring mechanism may include at least one of a group comprising a spike, a screw, a nail, a barb, and a hook. Each of the one or more rings may be connected by a cloth.

The central flow portion may comprise two or more telescoping members. In some embodiments, a first telescoping member of the two or more telescoping members has a first diameter. A second telescoping member of the two or more telescoping members may have a second diameter that is lesser/smaller than the first diameter and the second telescoping member may be configured to fit at least partially within a central opening of the first telescoping member. In some embodiments, the second telescoping member is configured to move with respect to the first telescoping member to adjust an amount of overlap between the first telescoping member and the second telescoping member. The second telescoping member may be configured to decrease the amount of overlap between the first telescoping member and the second telescoping member in response to expansion of the tissue wall. The first telescoping member and the second telescoping member may comprise one or more connection mechanisms configured to allow one-way movement of the second telescoping member.

In some embodiments, the central flow portion comprises a sheet of cloth configured to extend from the first anatomical chamber to the second anatomical chamber and stretch in response to expansion of the tissue wall. The sheet of cloth may be configured to form a cylindrical shape in the opening. The shunt may further comprise one or more anchoring mechanisms configured to anchor the sheet of cloth to a first side of the tissue wall. In some embodiments, the sheet of cloth forms a sac, is configured to at least partially cover the opening, and has one or more holes to allow blood flow through the sheet of cloth.

Some implementations of the present disclosure relate to a method comprising creating an opening in a tissue wall. The tissue wall is situated between a first anatomical chamber and a second anatomical chamber and the opening represents a blood flow path between the first anatomical chamber to the second anatomical chamber. The method further comprises placing a shunt at the opening. The shunt comprises a central flow portion configured to fit at least partially within the opening in the tissue wall, maintain the blood flow path from the first anatomical chamber to the second anatomical chamber, prevent in-growth of tissue within the opening, and expand in response to expansion of the tissue wall.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements. However, it should be understood that the use of similar reference numbers in connection with multiple drawings does not necessarily imply similarity between respective embodiments associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some embodiments or configurations.

FIG. 1 illustrates several access pathways for maneuvering guidewires and/or catheters in and around the heart to deploy expandable shunts in accordance with some embodiments.

FIG. 2 depicts a method for deploying expandable shunts in accordance with some embodiments.

FIG. 3A is a side view of an opening through a tissue wall for placement of a shunt in the opening in accordance with some embodiments.

FIG. 3B is a view from above (e.g., from the left atrium) of an opening through a tissue wall for placement of a shunt in the opening in accordance with some embodiments.

FIG. 4 illustrates a first expandable shunt implant in accordance with some embodiments.

FIG. 5 illustrates a second expandable shunt implant in accordance with some embodiments.

FIG. 6A illustrates a first expandable coiled shunt implant in accordance with some embodiments.

FIG. 6B illustrates a second expandable coiled shunt implant in accordance with some embodiments.

FIG. 7 illustrates an expandable ringed shunt implant in accordance with some embodiments.

FIG. 8 illustrates a telescoping shunt implant in accordance with some embodiments.

FIG. 9A illustrates a side-view of a cloth shunt implant in accordance with some embodiments.

FIG. 9B illustrates a view from above (e.g., from the left atrium) of a cloth shunt implant in accordance with some embodiments.

FIG. 10 is a flow diagram of an example of a process for delivering and/or anchoring an expandable shunt to a body of a person in accordance with some embodiments.

### DETAILED DESCRIPTION

The headings provided herein are for convenience only and do not necessarily affect the scope or meaning of the claimed invention.

### Overview

In vertebrate animals, the heart is a hollow muscular organ having four pumping chambers: the left and right atria and the left and right ventricles, each provided with its own one-way valve. The natural heart valves are identified as the aortic, mitral (or bicuspid), tricuspid and pulmonary, and are each mounted in an annulus comprising dense fibrous rings attached either directly or indirectly to the atrial and ventricular muscle fibers. Each annulus defines a flow orifice. The four valves ensure that blood does not flow in the wrong direction during the cardiac cycle; that is, to ensure that the blood does not back flow through the valve. Blood flows from the venous system and right atrium through the tricuspid valve to the right ventricle, then from the right ventricle through the pulmonary valve to the pulmonary artery and the lungs. Oxygenated blood then flows through the mitral valve from the left atrium to the left ventricle, and finally from the left ventricle through the aortic valve to the aorta/arterial system.

Heart failure is a common and potentially lethal condition affecting humans, with sub-optimal clinical outcomes often resulting in symptoms, morbidity and/or mortality, despite maximal medical treatment. In particular, “diastolic heart failure” refers to the clinical syndrome of heart failure occurring in the context of preserved left ventricular systolic function (ejection fraction) and in the absence of major valvular disease. This condition is characterized by a stiff left ventricle with decreased compliance and impaired relaxation, which leads to increased end-diastolic pressure. Approximately one third of patients with heart failure have diastolic heart failure and there are very few, if any, proven effective treatments.

Symptoms of diastolic heart failure are due, at least in a large part, to an elevation in pressure in the left atrium. Elevated Left Atrial Pressure (LAP) is present in several abnormal heart conditions, including Heart Failure (HF). In addition to diastolic heart failure, a number of other medical conditions, including systolic dysfunction of the left ventricle and valve disease, can lead to elevated pressures in the left atrium. Both Heart Failure with Preserved Ejection Fraction (HFpEF) and Heart Failure with Reduced Ejection Fraction (HFrEF) can exhibit elevated LAP. It has been hypothesized that both subgroups of HF might benefit from a reduction in LAP, which in turn reduces the systolic preload on the left ventricle, Left Ventricular End Diastolic Pressure (LVEDP). It could also relieve pressure on the pulmonary circulation, reducing the risk of pulmonary edema, improving respiration and improving patient comfort.

Pulmonary hypertension (PH) is defined as a rise in mean pressure in the main pulmonary artery. PH may arise from many different causes, but, in all patients, has been shown to increase mortality rate. A deadly form of PH arises in the very small branches of the pulmonary arteries and is known as Pulmonary Arterial Hypertension (PAH). In PAH, the cells inside the small arteries multiply due to injury or disease, decreasing the area inside of the artery and thickening the arterial wall. As a result, these small pulmonary arteries narrow and stiffen, causing blood flow to become restricted and upstream pressures to rise. This increase in pressure in the main pulmonary artery is the common connection between all forms of PH regardless of underlying cause. Despite previous attempts, there is a need for an improved way to reduce elevated pressure in the left atrium, as well as other susceptible heart chambers such as the pulmonary artery.

The present disclosure provides methods and devices that may allow for elevated LAP to be reduced by shunting blood from a first anatomical chamber (e.g., the left atrium) to a second anatomical chamber (e.g., the coronary sinus). While some embodiments herein may be described with respect to treating LAP and/or similar issues, the shunting devices and methods described may be used to treat other issues, including dialysis. Some embodiments involve a shunt defining an open pathway between the left atrium and the coronary sinus, although the method can be used to place a shunt between other cardiac chambers, such as between the pulmonary artery and right atrium. The terms “shunt” and/or “means for shunting” are used herein according to their plain and ordinary meaning and may refer to any medical implant configured to allow and/or facilitate blood flow from one part of a patient's body to another. The shunt may be configured to prevent initial collapse of the open pathway while also preventing in-growth of tissue at least at an inner surface of the open pathway. In some embodiments, the shunt may be expandable so as to be compressed, delivered via a low-profile sheath or tube, and expelled so as to resume its expanded state. Some methods may also include utilizing a deployment catheter that may first create a puncture in a tissue wall between the left atrium and the coronary sinus.

Moreover, in some embodiments, a shunt may be configured to expand post-delivery in response to expansion of the tissue wall. For example, some patients, and particularly HF patients, may experience amyloidosis, which is a protein disorder in which amyloid deposits in the heart can make the heart walls stiffen and/or increase in thickness. Shunt implants having a maximum tissue wall thickness specification may not be configured to accommodate some levels of tissue growth/expansion. For example, some shunt implants may have wall thickness specifications of approximately 4 mm. However, many amyloidosis patients can have tissue wall thickness that may continue to increase beyond 4 mm, therefore causing patency issues with shunt implants post-implantation. While it may be possible to at least partially constrain growth of the tissue walls, doing so may raise concerns of damaging the tissue. Accordingly, it may be advantageous for shunt implants to have an ability to expand and/or “grow” as tissue walls thicken.

Shunt implants described herein may therefore include a central flow portion that may be configured to expand at least longitudinally (e.g., a shunt implant passing through a tissue wall may expand in a direction of increasing thickness of the tissue wall) as a tissue wall expands and/or in response to tissue wall expansion. The central flow portion may incorporate various mechanical systems to allow expansion. Details of these methods, implants and deployment systems will be described below.

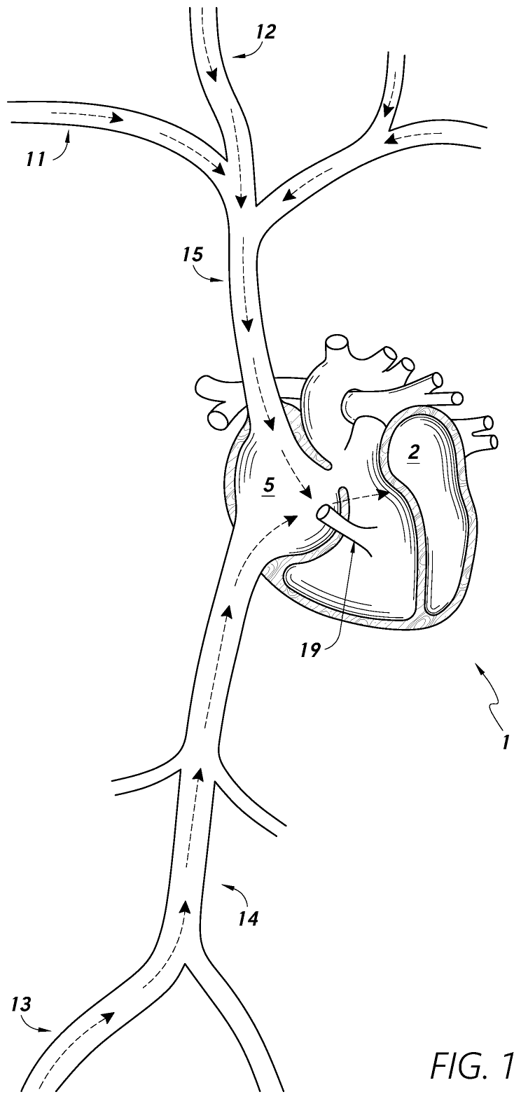

FIG. 1 illustrates several access pathways for maneuvering guidewires and catheters in and around the heart 1 to deploy expandable shunts of the present application. For instance, access may be from above via either the subclavian vein 11 or jugular vein 12 into the superior vena cava (SVC) 15, right atrium (RA) 5 and from there into the coronary sinus (CS) 19. Alternatively, the access path may start in the femoral vein 13 and through the inferior vena cava (IVC) 14 into the heart 1. Other access routes may also be used, and each typically utilizes a percutaneous incision through which the guidewire and catheter are inserted into the vasculature, normally through a sealed introducer, and from there the physician controls the distal ends of the devices from outside the body.

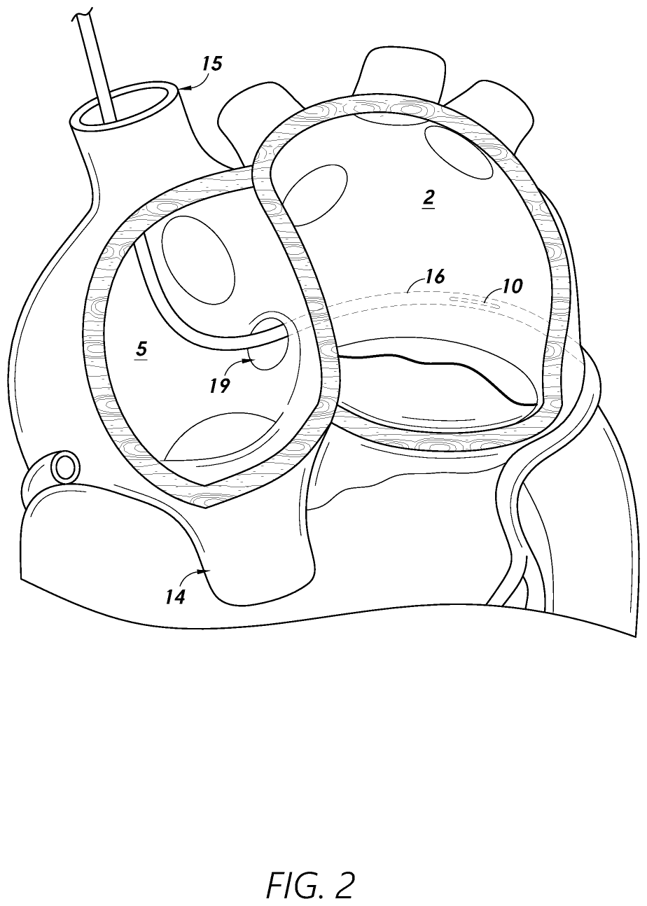

FIG. 2 depicts a method for deploying the expandable shunts described herein, wherein a guidewire is introduced through the subclavian or jugular vein via a catheter 16, through the SVC 15 and into the coronary sinus 19 for delivery of an implant device 10. Once the guidewire provides a path, an introducer sheath (not shown) may be routed along the guidewire and into the patient's vasculature, typically with the use of a dilator. FIG. 2 shows a deployment catheter 16 extending from the SVC 15 to the coronary sinus 19 of the heart 1, the deployment catheter 16 having been passed through the introducer sheath which provides a hemostatic valve to prevent blood loss.

In one embodiment, the deployment catheter 16 may be about 30 cm long, and the guidewire may be somewhat longer for ease of use. In some embodiments, the deployment catheter may function to form and prepare an opening in the wall of the left atrium 2, and a separate placement or delivery catheter will be used for delivery of an expandable shunt. In other embodiments, the deployment catheter may be used as the both the puncture preparation and shunt placement catheter with full functionality. In the present application, the terms “deployment catheter” or “delivery catheter” will be used to represent a catheter or introducer with one or both of these functions.

Since the coronary sinus 19 is largely contiguous around the left atrium 2, there are a variety of possible acceptable placements for the stent. The site selected for placement of the stent, may be made in an area where the tissue of the particular patient is less thick or less dense, as determined beforehand by non-invasive diagnostic means, such as a CT scan or radiographic technique, such as fluoroscopy or intravascular coronary echo (IVUS).

Some methods to reduce LAP involve utilizing a shunt between the left atrium 2 and the right atrium 5, through the interatrial septum therebetween. This is a convenient approach, as the two structures are adjacent and transseptal access is common practice. However, there may be a possibility of emboli travelling from the right side of the heart to the left, which presents a stroke risk. This event should only happen if the right atrium pressures go above left atrium pressures; primarily during discrete events like coughing, sneezing, Valsalva maneuver, or bowel movements. The anatomical position of the septum would naturally allow emboli to travel freely between the atria if a shunt was present and the pressure gradient flipped. This can be mitigated by a valve or filter element in the shunt, but there may still be risk that emboli will cross over.

Shunting to the coronary sinus 19 offers some distinct advantages, primarily that the coronary sinus 19 is much less likely to have emboli present for several reasons. First, the blood draining from the coronary vasculature into the right atrium 5 has just passed through capillaries, so it is essentially filtered blood. Second, the ostium of the coronary sinus 19 in the right atrium 5 is often partially covered by a pseudo-valve called the Thebesian Valve. The Thebesian Valve is not always present, but some studies show it is present in >60% of hearts and it would act as a natural “guard dog” to the coronary sinus to prevent emboli from entering in the event of a spike in right atrium pressure. Third, pressure gradient between the coronary sinus 19 and the right atrium 5 into which it drains is very low, meaning that emboli in the right atrium 5 is likely to remain there. Fourth, in the event that emboli do enter the coronary sinus 19, there will be a much greater gradient between the right atrium 5 and the coronary vasculature than between the right atrium 5 and the left atrium 2. Most likely emboli would travel further down the coronary vasculature until right atrium pressure returned to normal and then the emboli would return directly to the right atrium 5.

Some additional advantages to locating the shunt between the left atrium 2 and the coronary sinus 19 is that this anatomy is less mobile than the septum (it is more stable), it thus preserves the septum for later transseptal access for alternate therapies, and it could potentially have other therapeutic benefits. By diverting left atrial blood into the coronary sinus 19, sinus pressures may increase by a small amount. This would cause blood in the coronary vasculature to travel more slowly through the heart, increasing perfusion and oxygen transfer, which would be more efficient and also could help a dying heart muscle to recover. The preservation of transseptal access also is a very significant advantage because HF patients often have a number of other comorbidities like Atrial Fibrillation (AF) and Mitral Regurgitation (MR) and several of the therapies for treating these conditions require a transseptal approach.

A shunt may also be positioned between other cardiac chambers, such as between the pulmonary artery and right atrium 5. The shunt may be desirably implanted within the wall of the pulmonary artery using the deployment tools described herein, with the catheters approaching from above and passing through the pulmonary artery. As explained above, pulmonary hypertension (PH) is defined as a rise in mean pressure in the main pulmonary artery. Blood flows through the shunt from the pulmonary artery into the right atrium 5 if the pressure differential causes flow in that direction, which attenuates pressure and reduces damage to the pulmonary artery. The purpose is to attenuate pressure spikes in the pulmonary artery. The shunt may also extend from the pulmonary artery to other heart chambers (e.g., left atrium 2) and/or blood vessels. Although not preferred or shown, the shunt may further contain a one-way valve for preventing backflow, or a check valve for allowing blood to pass only above a designated pressure. The present application discloses a new expandable shunt. In some embodiments, an expandable shunt may be at least partially flexible and/or elastic in structure, which may advantageously simplify delivery processes for surgeons. For example, a shunt as described herein may be shaped and/or molded as desired/needed to fit openings through tissue walls in which the openings and/or tissue walls may have varying shapes and/or sizes. Moreover, the shunts may comprise any of a variety of types of anchoring arms and/or mechanisms which may be modified as needed to effectively anchor the shunts.

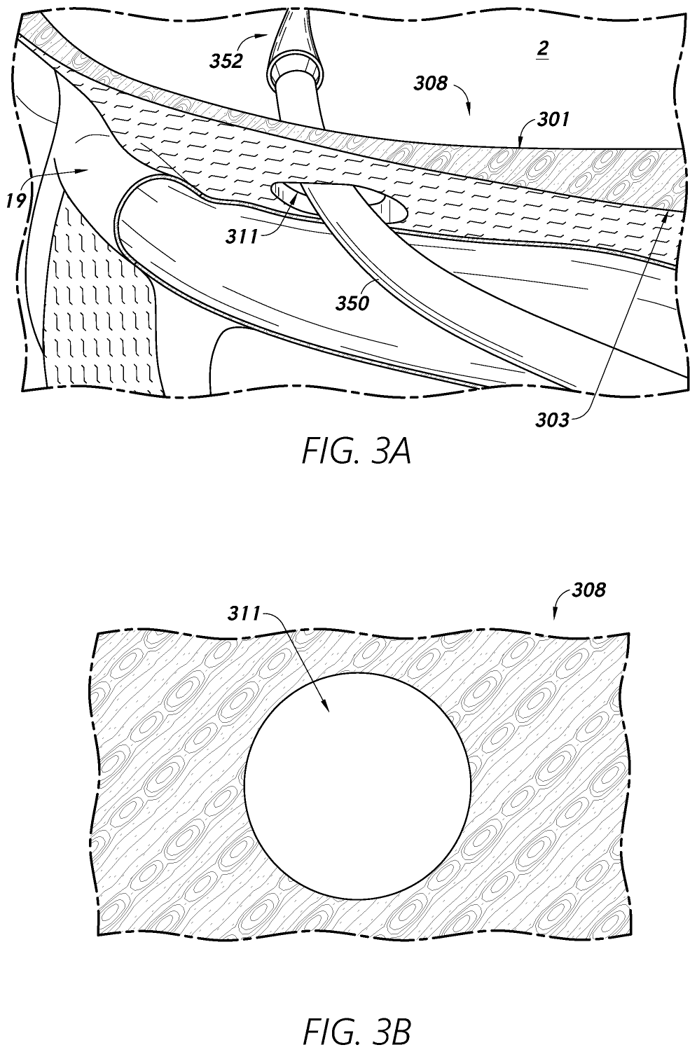

FIG. 3A is a side view and FIG. 3B is a view from above (e.g., from the left atrium 2) of an opening (i.e., puncture hole) 311 through a tissue wall 308 (e.g., between the coronary sinus 19 and the left atrium 2) for placement of a shunt in the opening 311. As shown in FIG. 3A, a shunt deployment or delivery catheter 350 may be advanced to the tissue wall 308 between two chambers (e.g., the coronary sinus 19 and the left atrium 2). The catheter 350 may have a soft and/or tapered distal tip 352. The delivery catheter 350 may be advanced through the opening 311 in the tissue wall 308 into, for example, the left atrium 2. The opening may be created in any of a variety of ways. One example method is the following.

Initially, a guidewire may be advanced, for example, from the right atrium into the coronary sinus 19 through its ostium or opening. A puncture catheter may be advanced over the guidewire. The puncture catheter may be introduced into the body through a proximal end of an introducer sheath. An introducer sheath may provide access to the particular vascular pathway (e.g., jugular or subclavian vein) and may have a hemostatic valve therein. While holding the introducer sheath at a fixed location, the surgeon can manipulate the puncture catheter to the implant site. A puncture sheath having a puncture needle with a sharp tip may be advanced along a catheter and punctured through the wall 8 into, for example, the left atrium 2. A puncture expander may be advanced along the guidewire and through the tissue wall 308 into the left atrium 2. The puncture expander may be, for example, an elongated inflatable balloon. The puncture expander may be inflated radially outward so as to widen the puncture through the tissue wall 308.

An expandable shunt may be delivered through a lumen of the catheter 350. During delivery, the expandable shunt may be in a collapsed configuration to facilitate delivery. For example, the shunt may be rolled, bent, twisted, and/or otherwise configured to have a minimal profile to facilitate delivery through the catheter 350. The shunt may be located in the annular space between an inner sheath and outer sheath of the catheter 350. An inner sheath may be retracted so that the shunt is placed in intimate engagement with the tissue wall 308. Radiopaque markers may be provided to facilitate positioning of the catheter 350 and/or shunt. By creating an opening between the left atrium 2 and the coronary sinus 19, blood can flow from the left atrium 2 (which is usually >8 mmHg) to the coronary sinus 19 (which is usually <8 mmHg). The shunt may be configured to attach/anchor to a first side 301 and/or a second side 303 of the tissue wall 308.

### Expandable Shunt Implants

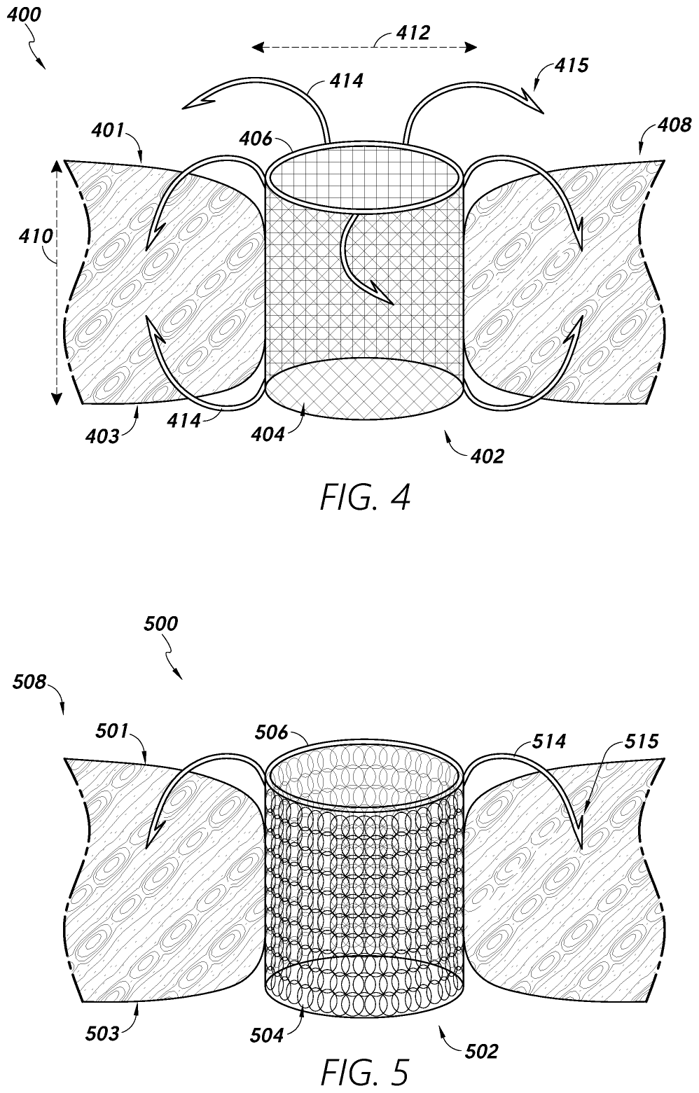

FIG. 4 illustrates a first expandable shunt implant in accordance with some embodiments. The first expandable shunt implant 400 may comprise a central flow portion 402 composed of a network of lines 404, which may include wires, sutures, strings, and/or various other elongate devices. One or more lines 404 may interact with each other in a weaving/interweaving and/or braiding pattern. For example, a first line may pass over a second line, under a third line, over a fourth line, and so on. Accordingly, the one or more lines 404 may have at least some flexibility such that a line 404 may be configured to bend over and/or under other lines 404. For example, one or more lines 404 may be composed of Nitinol and/or another material that is configured to at least partially bend and/or stretch.

The flow portion 402 may include any number of lines 404. In some embodiments, the flow portion 402 may comprise a single line 404 configured to interweave with itself. For example, the single line 404 may be configured to pass through (e.g., lace through) one or more devices such as rings 406 that may be configured to attach to and/or extend from the flow portion 402. A line 404 may pass through multiple rings 406 and/or may pass through a single ring 406 multiple times. A line 404 may enter the ring 406 at a first angle and exit the ring 406 at a second angle (e.g., approximately a 45-degree difference from the first angle).

By increasing the number of lines 404 and/or an amount of interweaving of the one or more lines 404, gaps between the lines 404 and/or different sections of a single line 404 may be minimized to improve prevention and/or reduction of in-growth of tissue.

Moreover, each of the lines 404 may have any thickness and may be designed to minimize gaps while maximizing expandability of the flow portion 402.

The flow portion 402 may comprise one or more rings 406 configured to attach to and/or extend from the network of lines 404. As shown in FIG. 4, the flow portion 402 may comprise a first ring 406 at a first end portion of the flow portion 402. For example, the first ring 406 may be situated at or near a first side 401 of a tissue wall. However, while only a single ring 406 is shown in FIG. 4, the flow portion 402 may comprise any number of rings 406. For example, a second ring 406 may be attached to the one or more lines 404 at a second end portion of the flow portion 402 near the second side 403 of the tissue wall 408. The flow portion 402 may be configured to be situated at least partially within an opening in the tissue wall (see, e.g., the opening 311 in FIGS. 3A and 3B). The tissue wall may have a first side 401 and a second side 403, and the opening may represent a gap through the tissue wall. A “thickness” of the tissue wall 408 may refer to a distance between the first side 401 and a second side 403 of the tissue wall 408. In other words, the “thickness” may represent a length of the tissue wall 408 along a longitudinal axis 410. As used herein, a “longitudinal” length may refer to a length perpendicular to (i.e., into, towards, and/or away from) a surface of a tissue wall 408. The opening through the tissue wall 408 may have a depth that is equal to the thickness of the tissue wall 408. In other words, the opening may pass entirely through a longitudinal length of the tissue wall 408. Moreover, the opening may have various widths. For example, opening may have a circular form (see, e.g., the opening 311 in FIGS. 3A and 3B) having a certain diameter. The “width” of the opening may refer to a length of the opening along a lateral axis 412. As used herein, a “lateral” length may refer to a length parallel to (i.e., along) a surface of the tissue wall 408.

At delivery, the flow portion 402 of the first expandable shunt implant 400 may have a length (measured along the longitudinal axis 410) that is approximately equal to a depth of the opening and/or a thickness of the tissue wall 408. Accordingly, a first ring 406 and/or a first end of the flow portion 402 may be approximately in-line along the longitudinal axis 410 with the first side 401 of the tissue wall 408 and/or a second ring 406 and/or a second end of the flow portion 402 may be approximately in-line along the longitudinal axis 410 with the second side 403 of the tissue wall 408. However, the first expandable shunt implant 400 may have a longitudinal length that is greater than the thickness of the tissue wall 408 (such that a first end and/or second end of the flow portion 402 extend out of the opening) or less than the thickness of the tissue wall 408 (such that a first end and/or second end of the flow portion 402 is/are situated within the opening.

The one or more lines 404 of the flow portion 402 may form a cylindrical or other shape to approximate a shape of the opening. In some embodiments, the opening may be widened in all directions approximately evenly from a puncture point to form an approximately circular opening having a certain diameter. Accordingly, the flow portion 402, including the one or more rings 406 and/or interconnected lines 404, may have an at least partially rounded and/or circular form around/about the longitudinal axis 410.

In some embodiments, the expandable shunt implant 400 may be in a compacted and/or otherwise expandable form at delivery. For example, at delivery, the one or more lines 404 may be situated relatively close together with minimal gaps between the one or more lines 404. As the tissue wall 408 expands (e.g., along the longitudinal axis 410), the one or more lines 404 may gradually separate and/or stretch to create a greater length (along the longitudinal axis 410) of the expandable shunt implant 400. In some embodiments, the one or more lines 404 may be configured to stretch in response to expansion of the tissue wall 408. For example, at delivery, the one or more lines 404 may be in a natural resting state and/or may be only minimally stretched. As the tissue wall 408 expands, at least some of the one or more lines 404 may stretch to create a greater length of the expandable shunt implant 400.

The expandable shunt implant 400 may comprise one or more anchoring arms 414, which may also be referred to as “means for anchoring,” configured to anchor to/into the tissue wall 408. While the expandable shunt implant 400 is shown having seven anchoring arms 414, the expandable shunt implant 400 may have any number of anchoring arms 414. In some embodiments, the expandable shunt implant 400 may comprise one or more anchoring arms 414 at a first end of the expandable shunt implant 400 (e.g., configured to anchor the first side 401 of the tissue wall 408) and/or one or more anchoring arms 414 at or near a second end of the expandable shunt implant 400 (e.g., configured to anchor to the second side 403 of the tissue wall 408). An anchoring arm 414 may attach to and/or extend from a ring 406 or one or more lines 404. For example, if the expandable shunt implant 400 does not include any rings 406, the anchoring arms 414 may attach to and/or extend from the lines 404.

Each of the anchoring arms 414 may comprise an anchoring mechanism 415 configured to penetrate, attach to, and/or otherwise anchor to the tissue wall 408. As shown in FIG. 4, an anchoring mechanism 415 may include a barb. However, suitable mechanisms 415 may include one or more of hooks, needles, screws, nails and/or other devices.

In some embodiments, each of the lines 404, rings 406, and/or anchoring arms 414 may be composed of a common material or different materials. In some embodiments, any of the lines 404, rings 406, and/or anchoring arms 414 may be composed of Nitinol and/or other metal, plastic, polymer, and/or other material. In some embodiments, a ring 406 may have an at least partially rigid structure to provide an amount of stability to the expandable shunt implant 400. For example, the one or more rings 406 may be configured to hold a pre-determined form even as the expandable shunt implant 400 expands. In this way, the one or more rings 406 may be configured to prevent unnecessary damage to the tissue wall 408. For example, one or more anchoring arms 414 may extend from and/or attach to a ring 406. Due at least in part to the rigid structure of the ring 406, the flow portion 402 may provide a consistent level of pressure and/or may provide a consistent orientation with respect to the one or more anchoring arms 414.

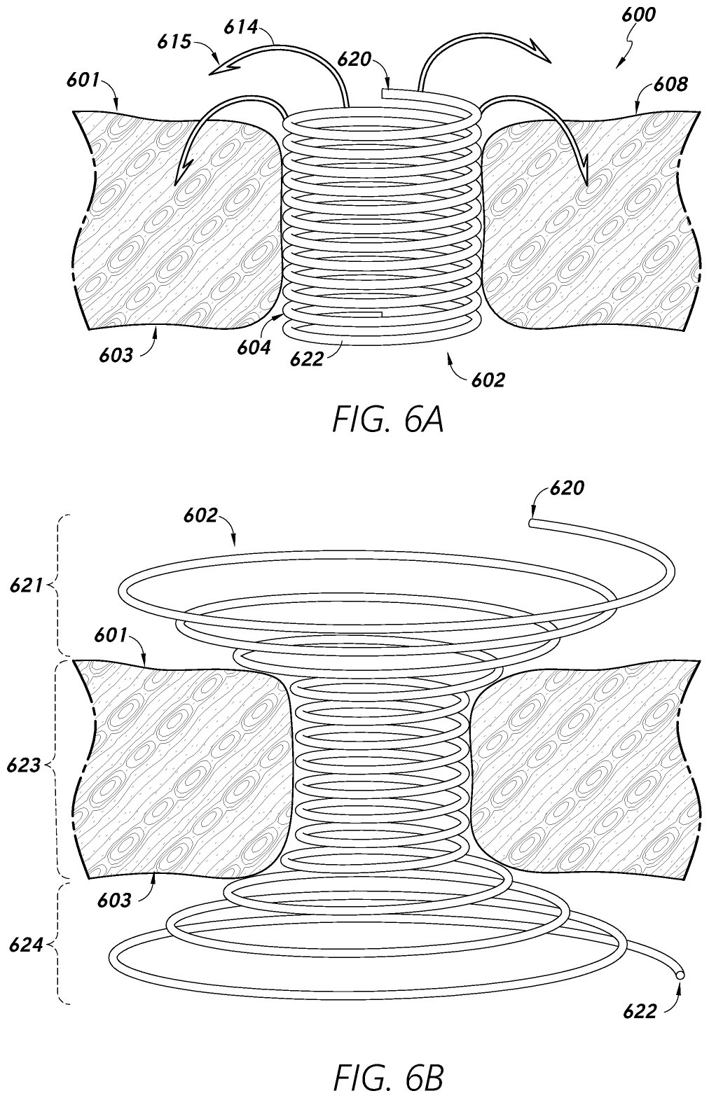

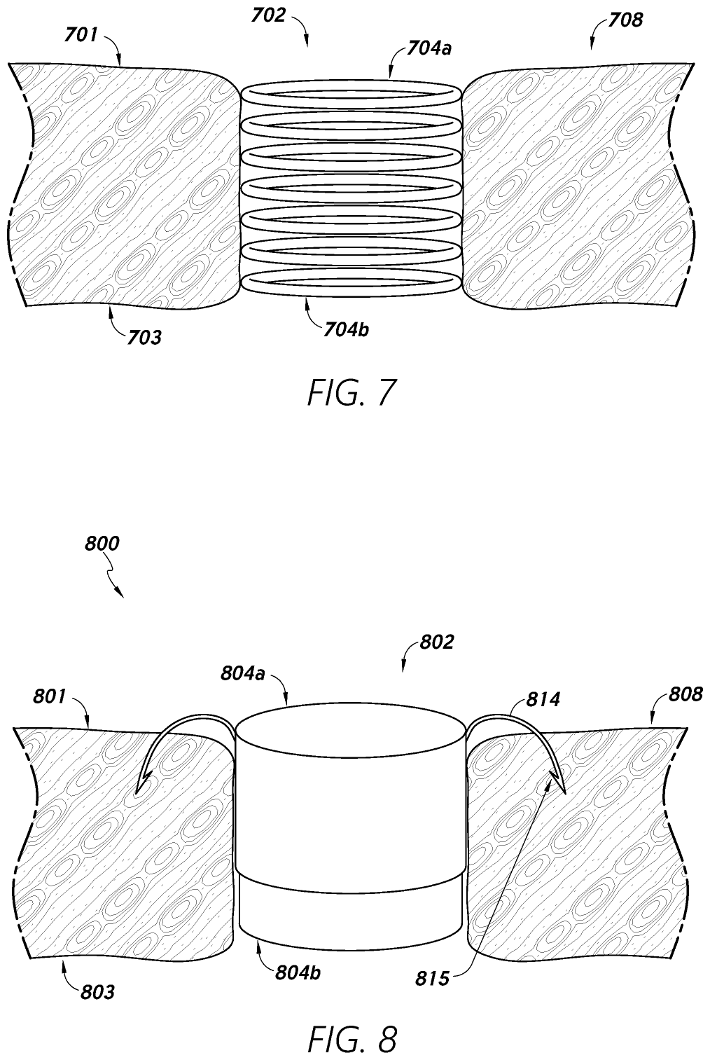

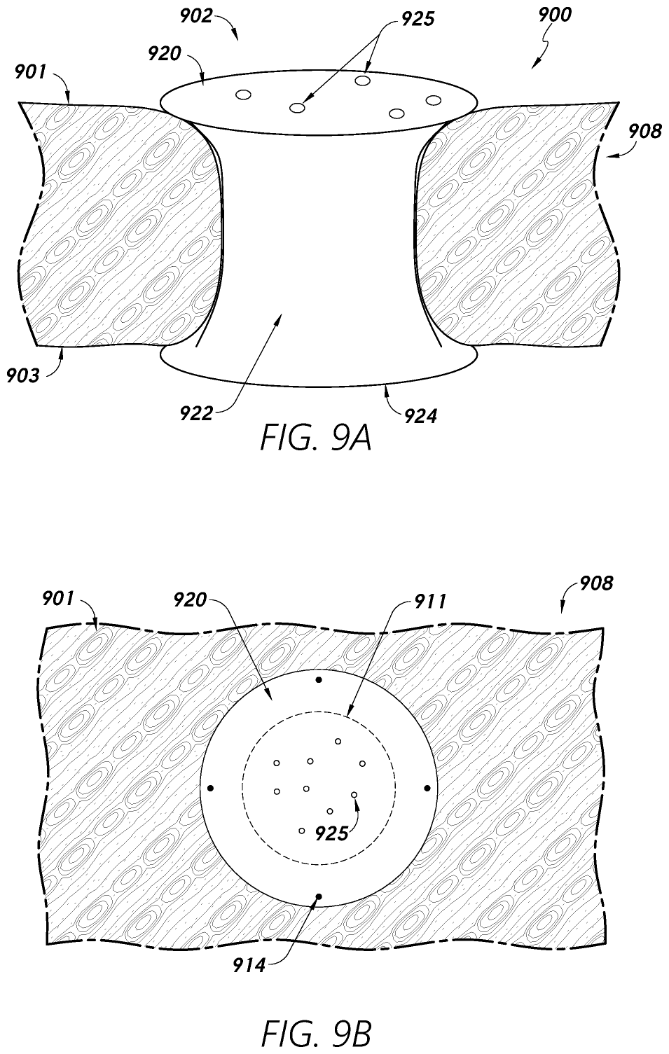

Various features of the shunt implant 400, including the central flow portion 402 and/or anchoring arms 414 described herein may be applied to the shunt devices described and/or illustrated in other figures of the present application. For example, any description with respect to the shunt implant 400 illustrated in FIG. 4 may be similarly applied to the shunt implant 500 in FIG. 5, the shunt implant 600 in FIGS. 6A and/or 6B, the shunt implant in FIG. 7, the shunt implant in FIG. 8, and/or the shunt implant in FIGS. 9A and 9B described herein. Moreover, while other shunts shown and/or described with respect to other figures may not include lines 404 and/or rings 406 as shown in FIG. 4, it will be understood that lines 404 and/or rings 406 may be added to the shunts described with respect to other figures. Similarly, the various features described with respect to other figures herein may be added to the shunt implant 400 of FIG. 4 and/or other figures herein even if not depicted in and/or described with respect to each figure. While the shunt implant 400 is shown including both a central flow portion 402 and anchoring arms 414, the shunt implant 400 may in some embodiments not include anchoring arms 414.

FIG. 5 illustrates a second expandable shunt implant in accordance with some embodiments. The second expandable shunt implant 500 may comprise a central flow portion 502 composed of a network of chains 504, which may include wires, sutures, strings, and/or various other devices. Each chain 504 may be configured to interlock with one or more other chains 504 to form a “chainmail” pattern of chains 504. While the chains 504 are shown in FIG. 5 having a generally circular shape, each chain 504 may have any suitable shape and/or size. For example, a chain 504 may have a triangular, octagonal, pentagonal, rectangular, or other shape. Each chain 504 may interlock with any number of other chains 504. For example, a first chain 504 at an end of the flow portion 502 (e.g., connected to a ring 506) may be interlocked with five other chains 504 (e.g., one chain 504 on a right side of the first chain 504, one chain on a left side of the first chain 504, and three chains below the first chain 504). In other words, five chains 504 may pass through the hole of the first chain 504. In another example, a first chain 504 not at an end of the flow portion 502 may be connected to eight chains 504 (e.g., three chains 504 above the first chain 504, one chain 504 on a right side of the first chain 504, one chain on a left side of the first chain 504, and three chains below the first chain 504).

The flow portion 502 may further comprise one or more rings 506 configured to attach to and/or extend from the network of chains 504. For example, a ring 506 may pass through holes of one or more chains 504. As shown in FIG. 5, the flow portion 502 may comprise a first ring 506 at a first end of the flow portion 502. For example, the first ring 506 may be situated at or near a first side 501 of a tissue wall 508. The flow portion 502 may be situated at least partially within an opening in the tissue wall. The tissue wall 508 may have a first side 501 and a second side 503, and the opening may represent a gap through the tissue wall. The opening through the tissue wall 508 may have a depth that is equal to the thickness of the tissue wall 508. Moreover, the opening may have various widths. For example, the opening may have a generally circular form (see, e.g., the opening 311 in FIGS. 3A and 3B) having a certain diameter.

At delivery, the flow portion 502 of the second expandable shunt implant 500 may have a longitudinal length that is approximately equal to a depth of the opening and/or a thickness of the tissue wall 508. Accordingly, a first ring 506 and/or a first end of the flow portion 502 may be approximately in-line along a longitudinal axis with the first side 501 of the tissue wall 508 and/or a second ring 506 and/or a second end of the flow portion 502 may be approximately in-line along the longitudinal axis with the second side 503 of the tissue wall 508. However, the second expandable shunt implant 500 may have a longitudinal length that is greater than the thickness of the tissue wall 508 (such that a first end and/or second end of the flow portion 502 extend out of the opening) or less than the thickness of the tissue wall 508 (such that a first end and/or second end of the flow portion 502 is/are situated within the opening.

The one or more chains 504 of the flow portion 502 may form a cylindrical or other shape to approximate a shape of the opening. In some embodiments, an opening may be widened in all directions approximately evenly from a puncture point to form a circular opening having a certain diameter. Accordingly, the flow portion 502, including the one or more rings 506 and/or interconnected chains 504, may have an at least partially rounded and/or circular form around a longitudinal axis.

In some embodiments, the expandable shunt implant 500 may be in a compacted and/or otherwise expandable form at delivery. For example, at delivery, the one or more chains 504 may be situated relatively close together with minimal separation between the one or more chains 504. As the tissue wall 508 expands (e.g., longitudinally), the one or more chains 504 may gradually separate to create a greater longitudinal length of the expandable shunt implant 500. In some embodiments, the one or more chains 504 may be configured to stretch in response to expansion of the tissue wall 508. For example, at delivery, the one or more chains 504 may be in a natural resting state and/or may be only minimally stretched. As the tissue wall 508 expands, at least some of the one or more chains 504 may stretch to create a greater length of the expandable shunt implant 500. In some embodiments, the flow portion 502 may comprise one or more restraining mechanisms to prevent expansion of the flow portion 502 before corresponding expansion of the tissue wall 508. For example, two or more chains 504 may be held close together by a suture, wire, or similar device. As the tissue wall 508 expands, the pressure exerted on the restraining mechanism(s) may increase to a level that the restraining mechanism(s) breaks and/or stretches to allow a greater level of separation between the two or more chains 504.

The expandable shunt implant 500 may comprise one or more anchoring arms 514 configured to anchor into the tissue wall 508. While the expandable shunt implant 500 is shown having two anchoring arms 514, the expandable shunt implant 500 may have any number of anchoring arms 514. In some embodiments, the expandable shunt implant 500 may comprise one or more anchoring arms 514 at a first end of the expandable shunt implant 500 (e.g., configured to anchor the first side 501 of the tissue wall 508) and/or one or more anchoring arms 514 at or near a second end of the expandable shunt implant 500 (e.g., configured to anchor to the second side 503 of the tissue wall 508). An anchoring arm 514 may attach to and/or extend from a ring 506 or one or more chains 504. For example, if the expandable shunt implant 500 does not include any rings 506, the anchoring arms 514 may attach to and/or extend from the chains 504.

Each of the anchoring arms 514 may comprise an anchoring mechanism 515 configured to penetrate, attach to, and/or otherwise anchor to the tissue wall 508. As shown in FIG. 5, an anchoring mechanism 515 may include a barb. However, suitable mechanisms 515 may include one or more of hooks, needles, screws, nails and/or other devices.

In some embodiments, each of the chains 504, rings 506, and/or anchoring arms 514 may be composed of a common material or different materials. In some embodiments, any of the chains 504, rings 506, and/or anchoring arms 514 may be composed of Nitinol and/or other metal, plastic, polymer, or other material. In some embodiments, a ring 506 may have an at least partially rigid structure to provide a level of stability to the expandable shunt implant 500. For example, the one or more rings 506 may be configured to hold a pre-determined form even as the expandable shunt implant 500 expands. In this way, the one or more rings 506 may be configured to prevent unnecessary damage to the tissue wall 508. For example, one or more anchoring arms 514 may extend from and/or attach to a ring 506. Due at least in part to the rigid structure of the ring 506, the flow portion 502 may provide a consistent level of pressure and/or may provide a consistent orientation with respect to the one or more anchoring arms 514.

FIGS. 6A and 6B illustrate expandable coiled shunt implants in accordance with some embodiments. A coiled shunt implant 600 may comprise a central flow portion 602 composed of one or more coiled lines 604. In some embodiments, the flow portion 602 and/or a single coiled line 604 may extend at least from a first side 601 of a tissue wall 608 to a second side 603 of the tissue wall 608. The flow portion 602 may be situated at least partially within an opening in the tissue wall 608.

At delivery, the flow portion 602 of the coiled shunt implant 600 may have a longitudinal length that is approximately equal to a depth of the opening and/or a thickness of the tissue wall 608. Accordingly, a first end 620 of the flow portion 602 may be approximately in-line along a longitudinal axis with the first side 601 of the tissue wall 608 and/or a second end 622 of the flow portion 602 may be approximately in-line along the longitudinal axis with the second side 603 of the tissue wall 608. However, the coiled shunt implant 600 may have a longitudinal length that is greater than the thickness of the tissue wall 608 (such that the first end 620 and/or second end 622 of the flow portion 602 extend out of the opening) or less than the thickness of the tissue wall 608 (such that the first end 620 and/or second end 622 of the flow portion 602 is/are situated within the opening).

The one or more lines 604 of the flow portion 602 may form a cylindrical or other shape to approximate a shape of the opening. In some embodiments, an opening in the tissue wall 608 may be widened in all directions approximately evenly from a puncture point to form a circular opening having a certain diameter. Accordingly, the flow portion 602, including the one or more lines 604, may have an at least partially rounded and/or circular form around a longitudinal axis.

In some embodiments, the expandable shunt implant 600 may be in a compacted and/or otherwise expandable/unexpanded form at delivery. For example, at delivery, the one or more lines 604 may form a set of relatively tight coils with minimal separation between the coils of the one or more lines 604. As the tissue wall 608 expands (e.g., longitudinally), the set of coils may gradually expand/separate to create a greater longitudinal length of the coiled shunt implant 600. In some embodiments, the one or more lines 604 may have a feature of elasticity such that when the one or more lines 604 expand, the one or more lines 604 may naturally exert a force to return to a resting (e.g., unexpanded) state.

The coiled shunt implant 600 may comprise one or more anchoring arms 614 configured to anchor into the tissue wall 608. While the coiled shunt implant 600 is shown having four anchoring arms 614, the coiled shunt implant 600 may have any number of anchoring arms 614. In some embodiments, the coiled shunt implant 600 may comprise one or more anchoring arms 614 at or near the first end 620 of the coiled shunt implant 600 (e.g., configured to anchor the first side 601 of the tissue wall 608) and/or one or more anchoring arms 614 at or near the second end 622 of the coiled shunt implant 600 (e.g., configured to anchor to the second side 603 of the tissue wall 608). An anchoring arm 614 may attach to and/or extend from one or more lines 604.

Each of the anchoring arms 614 may comprise an anchoring mechanism 615 configured to penetrate, attach to, and/or otherwise anchor to the tissue wall 608. As shown in FIG. 6A, an anchoring mechanism 615 may include a barb. However, suitable mechanisms 615 may include one or more of hooks, needles, screws, nails and/or other devices.

In some embodiments, each of the lines 604 and/or anchoring arms 614 may be composed of a common material or different materials. In some embodiments, any of the lines 604 and/or anchoring arms 614 may be composed of Nitinol and/or other metal, plastic, polymer, or other material.

FIG. 6B shows a coiled shunt implant 600 in which the first end 620 of the flow portion 602 may extend beyond the first side 601 of the tissue wall 608 and into a first anatomical chamber. The second end 622 may extend beyond the second side 603 of the tissue wall 608 and into a second anatomical chamber. For example, first section 621 of the flow portion 602 may be beyond the first side 601 of the tissue wall 608, a second section 623 of the flow portion 602 may be within the tissue wall 608, and/or a third section 624 may be beyond a second side 603 of the tissue wall 608. In some embodiments, the flow portion 602 may have a varying diameter. For example, the flow portion 602 may have a minimal and/or fixed diameter at the second section 623. The flow portion 602 may expand to a greater diameter at the first section 621 and/or at the third section 624. In some embodiments, the diameter of the flow portion 602 may gradually increase between approximately the first side 601 of the tissue wall 608 and the first end 620 of the flow portion 602. Similarly, the diameter of the flow portion 602 may gradually increase between approximately the second side 603 of the tissue wall 608 and the second end 622 of the flow portion 602. However, in some embodiments, the flow portion 602 may have a generally fixed and/or maximal diameter at the first section 621 and/or at the third section 624.

The diameter of the flow portion 602 at the first section 621 and/or at the third section 624 may be greater than a diameter of the opening in the tissue wall 608. In this way, at least a portion of the first section 621 and/or the third section 624 may be prevented from entering the opening of the tissue wall 608 and the flow portion 602 may be held in place by the tissue wall 608. Accordingly, the coiled shunt implant 600 may not include any anchoring arms 614, as the coiled shunt implant 600 may be anchored to the tissue wall 608 to prevent dislodging of the flow portion 602 without requiring anchoring arms 614.

The diameter of the second section 623 of the flow portion 602 may be approximately equal to a diameter of the opening in the tissue wall 608. Accordingly, the second section 623 of the flow portion 602 may be configured to press against the tissue wall 608 to prevent in-growth of tissue at the opening. At least the second section 623 (and/or the first section 621 and/or third section 624) may be configured to expand longitudinally in response to an increase of thickness of the tissue wall 608. As the tissue wall 608 thickens, coils of the flow portion 602 may separate to increase a longitudinal length of the flow portion 602. In some embodiments, the flow portion 602 may include a relatively large number of coils such that the flow portion 602 may be configured to increase in longitudinal length without requiring a high degree of separation between each set of coils. In this way, separation between the coils may be minimized even during expansion to prevent in-growth of the tissue and thereby to maintain a shape and/or size of the opening in the tissue wall 608.

FIG. 7 illustrates an expandable ringed shunt implant in accordance with some embodiments. A ringed shunt implant may comprise a central flow portion 702 composed of one or more rings 704. In some embodiments, the flow portion 702 may extend at least from a first side 701 of a tissue wall 708 to a second side 703 of the tissue wall 708. The flow portion 702 may be situated at least partially within an opening in the tissue wall 708. While the central flow portion 702 is shown comprising seven rings 704, the central flow portion 702 may comprise any number of rings 704.

At delivery, the flow portion 702 of the ringed shunt implant may have a longitudinal length that is approximately equal to a depth of the opening and/or a thickness of the tissue wall 708. Accordingly, a first ring 704a of the flow portion 702 may be approximately in-line along a longitudinal axis with the first side 701 of the tissue wall 708 and/or a second ring 704b of the flow portion 702 may be approximately in-line along the longitudinal axis with the second side 703 of the tissue wall 708. However, the ringed shunt implant may have a longitudinal length that is less than the thickness of the tissue wall 708 (such that the first ring 704a and/or second ring 704b of the flow portion 702 is/are situated within the opening).

Each of the one or more rings 704 may have a circular and/or elliptical shape to approximate a shape of an opening in the tissue wall 708. The one or more rings 704 may be configured to press against an inner surface of the tissue wall 708 and/or penetrate the tissue wall 708. In some embodiments, one or more rings 704 may have spikes and/or similar features configured to penetrate and/or anchor to the inner surface of the tissue wall 708 to hold the rings 704 in place.

In some embodiments, the ringed shunt implant may be in a compacted and/or otherwise expandable/unexpanded form at delivery. For example, at delivery, the one or more rings 704 may have a minimal distance of separation from each other. As the tissue wall 708 expands (e.g., longitudinally), the rings may gradually separate to create a greater longitudinal length of the ringed shunt implant.

In some embodiments, the one or more rings 704 may be connected via one or more wires, cloths, and/or similar devices. For example, a cloth or similar material having an approximately cylindrical form may surround and/or attach to the one or more rings 704. In this way, the cloth may fill gaps between the one or more rings 704 to prevent in-growth of tissue between the rings 704.

The ringed shunt implant may comprise one or more anchoring arms configured to anchor into the tissue wall 708. For example, the ringed shunt implant may comprise one or more anchoring arms attached to and/or extending from the first ring 704a of the ringed shunt implant (e.g., configured to anchor the first side 701 of the tissue wall 708) and/or one or more anchoring arms attached to and/or extending from the second ring 704b of the ringed shunt implant (e.g., configured to anchor to the second side 703 of the tissue wall 708).

In some embodiments, each of the rings 704 may be composed of a common material or different materials. In some embodiments, any of the rings 704 may be composed of Nitinol and/or other metal, plastic, polymer, or other material. One or more of the rings 704 may be composed of Nitinol or other shape-memory material and may be shape-set to naturally assume a greater diameter than the opening in the tissue wall 708 such that the rings 704 may press against the inner surface of the opening to hold itself in place. For example, a ring 704 may comprises a non-continuous line that may be configured to coil in response to force. One or more rings 704 may be configured to be compressed to have a smaller diameter when placed into the opening in the tissue wall 708. In some embodiments, the rings 704 and/or anchoring arms may be composed of and/or coated in Carbothane and/or a similar material (e.g., a polymer) configured to prevent and/or inhibit in-growth of the tissue.

FIG. 8 illustrates a telescoping shunt implant in accordance with some embodiments. The telescoping shunt implant 800 may comprise a central flow portion 802 composed of one or more telescoping members 804. While the telescoping members 804 are shown in FIG. 8 having a cylindrical shape, each telescoping member 804 may have any suitable shape and/or size. In some embodiments, a first telescoping member 804a may have a greater diameter/width than a second telescoping member 804b, such that the second telescoping member 804b may be configured to fit at least partially within/into a central opening/area of the first telescoping member 804a. While FIG. 8 shows only two telescoping members 804, the flow portion 802 may include more than two telescoping members 804.

As shown in FIG. 8, an end of the first telescoping member 804a may be configured to be situated at or near a first side 801 of a tissue wall 808. The flow portion 802 may be situated at least partially within an opening in the tissue wall. The tissue wall 808 may have a first side 801 and/or a second side 803, and the opening may represent a gap through the tissue wall. The opening through the tissue wall 808 may have a depth that is equal to the thickness of the tissue wall 808. Moreover, the opening may have various widths. For example, the opening may have a circular form (see, e.g., the opening 311 in FIGS. 3A and 3B) having a certain diameter.

At delivery, the flow portion 802 of the telescoping shunt implant 800 may be configured to have a longitudinal length that is approximately equal to a depth of the opening and/or a thickness of the tissue wall 808. Accordingly, an end of the first telescoping member 804a may be configured to be situated approximately in-line along a longitudinal axis with the first side 801 of the tissue wall 808 and/or an end of the second telescoping member 804b may be configured to be situated approximately in-line along the longitudinal axis with the second side 803 of the tissue wall 808. However, the telescoping shunt implant 800 may have a longitudinal length that is greater than the thickness of the tissue wall 808 (such that a first end and/or second end of the flow portion 802 may be configured to extend out of the opening) or less than the thickness of the tissue wall 808 (such that a first end and/or second end of the flow portion 802 may be configured to be situated within the opening).

The two or more telescoping members 804 of the flow portion 802 may form a cylindrical or other shape to approximate a shape of the opening in the tissue wall 808. In some embodiments, an opening may be widened in all directions approximately evenly from a puncture point to form an elliptical (e.g., circular) opening having a certain diameter. Accordingly, the flow portion 802, including the two or more telescoping members 804, may have an at least partially rounded and/or circular form around a longitudinal axis.

The telescoping shunt implant 800 may be in a compacted and/or otherwise expandable form at delivery. At delivery, the two or more telescoping members 804 may have a maximal amount of overlap. For example, the second telescoping member 804b may be situated entirely within a central (e.g., at least partially hollow) area of the first telescoping member 804a. As the tissue wall 808 expands (e.g., longitudinally), the amount of overlap between the two or more telescoping members 804 may gradually decrease to create a greater longitudinal length of the telescoping shunt implant 800. For example, the first telescoping member 804a may be configured to move with respect to the second telescoping member 804b and/or the second telescoping member 804a may be configured to move with respect to the first telescoping member 804b to adjust an amount of overlap between the telescoping members 804.

In some embodiments, each telescoping member 804 may be attached to and/or may extend from at least one other telescoping member. For example, the first telescoping member 804a may be attached to the second telescoping member 804b. In some embodiments, an attachment may be a slidable attachment. For example, the first telescoping member 804a may comprise a guide track configured to fit a peg, notch or similar mechanism. The second telescoping member 804b may comprise a peg, notch, or similar mechanism configured to fit into/onto the guide track of the first telescoping member 804a. Accordingly, the second telescoping member 804b may be configured to slide with respect to the first telescoping member 804a or vice versa. In some embodiments, a slidable attachment between multiple telescoping member 804 may involve use of various stoppers (e.g., cords, pegs, notches, teeth, etc.) configured to at least temporarily prevent and/or resist movement of the telescoping members 804 with respect to each other. For example, the second telescoping member 804b may be configured to slide along a guide track of the first telescoping member 804a and may interact with one or more stoppers while sliding along the guiding track. The stoppers may be configured to stop and/or slow the second telescoping member 804b temporarily and/or until a sufficient force is applied for the second telescoping member 804b to break and/or push through the stopper. In this way, longitudinal expansion of the flow portion 802 may be controlled and/or divided into stages to match and/or approximate expansion of the flow portion 802 to increasing thickness of the tissue wall 808. Moreover, the telescoping members 804 may include other attachment mechanisms in addition to and/or in place of a guiding track and/or corresponding pegs/notches. For example, the first telescoping member 804a may comprise a round gear and/or linear rack with teeth configured to interact with one or more pawls or similar mechanisms of the second telescoping member 804b to create a ratcheting connection between the telescoping members 804. One or more teeth of the gear and/or rack may be asymmetrical and/or may be partially sloped on a first edge with a steeper slope on a second edge. In this way, the pawl or similar mechanism of the second telescoping member 804b may be configured to move more easily in one direction (e.g., decreasing an amount of overlap between the first telescoping member 804a and the second telescoping member 804b) than in a second direction (e.g., increasing the amount of overlap between the first telescoping member 804a and the second telescoping member 804b).

The telescoping members 804 may be configured to move in response to expansion of the tissue wall 808. In some embodiments, the flow portion 802 may comprise one or more connection/restraining mechanisms to prevent expansion of the flow portion 802 before corresponding expansion of the tissue wall 808. For example, two or more telescoping members 804 may be held with maximal overlap by a suture, clamp, or similar device. As the tissue wall 808 expands, the pressure exerted on the restraining mechanism(s) may increase to a level that the restraining mechanism(s) breaks and/or stretches to allow extension of the flow portion 802 in which an amount of overlap between the two or more telescoping members 804 decreases.

In some embodiments, the telescoping shunt implant 800 may include one or more pegs, notches, and/or similar mechanisms to allow the flow portion 802 to expand in levels. For example, the first telescoping member 804a may comprise one or more notches configured to corresponding pegs extending from the second telescoping member 804b. At delivery a first peg extending from the second telescoping member 804b may be situated within a first notch of the first telescoping member 804a. As the tissue wall 808 expands, the first peg may slide along the first telescoping member 804a and settle into a second notch of the first telescoping member 804a. When a peg (or similar mechanism of the second telescoping member 804b interacts with a notch (or similar mechanism) of the first telescoping member 804a, there may be a resistive force to prevent movement of the second telescoping member 804b with respect to the first telescoping member 804a until a sufficient force (e.g., expansion of the tissue wall 808) is applied to the second telescoping member 804b and/or first telescoping member 804a. In some embodiments, the mechanisms may be configured to allow one-way movement of the telescoping members 804 (i.e., movement in only one direction), similar to a ratchet.

The telescoping shunt implant 800 may comprise one or more anchoring arms 814 configured to anchor into the tissue wall 808. While the telescoping shunt implant 800 is shown having two anchoring arms 814, the telescoping shunt implant 800 may have any number of anchoring arms 814. In some embodiments, the telescoping shunt implant 800 may comprise one or more anchoring arms 814 at a first end of the telescoping shunt implant 800 (e.g., configured to anchor the first side 801 of the tissue wall 808) and/or one or more anchoring arms 814 at or near a second end of the telescoping shunt implant 800 (e.g., configured to anchor to the second side 803 of the tissue wall 808). Anchoring arms 814 may attach to and/or extend from the two or more telescoping members 804.

Each of the anchoring arms 814 may comprise an anchoring mechanism 815 configured to penetrate, attach to, and/or otherwise anchor to the tissue wall 808. As shown in FIG. 8, an anchoring mechanism 815 may include a barb. However, suitable mechanisms 815 may include one or more of hooks, needles, screws, nails and/or other devices.

In some embodiments, each of the telescoping members 804 and/or anchoring arms 814 may be composed of a common material or different materials. In some embodiments, any of the telescoping members 804 and/or anchoring arms 814 may be composed of Nitinol and/or other metal, plastic, polymer, or other material.

FIGS. 9A and 9B illustrate a cloth shunt implant 900 in accordance with some embodiments. FIG. 9A shows a side view of the cloth shunt implant 900. The cloth shunt implant 900 may comprise a central flow portion 902 (having a first section 920, a second section 922, and/or third section 924) composed of a single continuous sheet of cloth or one or more non-continuous sheets of cloth. As used herein, “cloth” may refer to any elastic and/or flexible material that is capable to being stretched, molded, and/or otherwise shaped in response to various forces. The central flow portion 902 may comprise a piece of cloth in the form of a sac, tube, bag, or sheet. For example, the central flow portion 902 may comprise a sac having a continuous structure in which the central flow portion 902 does not have any edges, corners, etc. The cloth may be composed of an elastic material such that the cloth may be configured to stretch in response to force and/or return to a pre-defined form when force is removed. In some embodiments, the central flow portion 902 may have an at least partially hollow interior which may be completely surrounded by cloth. The central flow portion 902 may be at least partially amorphous such that the central flow portion 902 may be shaped to form a variety of shapes and/or may be stretched to have a variety of sizes. The central flow portion 902 may be configured to stretch longitudinally (i.e., increasing a distance between the first section 920 and the third section 924) in response to expansion and/or growth of the tissue wall 908.

As shown in FIG. 9A, a first section 920 of the flow portion 902 may be configured to be situated at or near a first side 901 of the tissue wall 908, a second section 922 of the flow portion 902 may be configured to be situated within an opening of the tissue wall 908, and a third section 924 of the flow portion 902 may be configured to be situated at or near a second side 903 of the tissue wall 908. In some embodiments, the flow portion 902 may be configured to at least partially cover the opening in the tissue wall. For example, the first section 920 may be configured to at least partially cover the opening at the first side 901 of the tissue wall 908 and/or the third section 924 may be configured to at least partially cover the opening at the second side 903 of the tissue wall 908. The first section 920 and/or the third section 924 may be configured to at least partially cover the opening at a resting state and/or may be configured to be stretched to a sufficient extent to cover the opening.

In some embodiments, the cloth shunt implant 900 may be configured to define and/or maintain a flow path through the tissue wall 908. The central flow portion 902 (e.g., the first section 920 and/or the third section 924) may be composed of a material with a breathable structure that may allow flow through the central flow portion 902. For example, the central flow portion 902 may be composed of a material that comprises multiple weaved fibers with small gaps between the fibers. Accordingly, blood may be capable of flowing through the central flow portion 902. In some embodiments, the flow portion 902 (e.g., the first section 920 and/or the third section 924) may have one or more holes 925 configured to allow blood to flow through the flow portion 902. Each of the holes 925 may have a sufficient size to allow blood flow. The flow portion 902 may have any number of holes 925 and/or the holes 925 may have any size and/or shape. The holes 925 may be positioned at points in the first section 920 and/or the third section 924 configured to be in line with the opening through the tissue wall 908. Accordingly, blood flow through the holes 925 may pass through the central flow portion 902 and through the opening.

FIG. 9B shows an overhead view (e.g., viewed from the left atrium) of the cloth shunt implant 900 on the first side 901 of the tissue wall 908. As shown in FIG. 9B, an opening 911 may be created in the tissue wall 908. The opening 911 is shown in FIG. 9B as a dashed line to represent the positioning of the opening 911 with respect to the central flow portion 902. The opening 911 may be at least partially covered by the central flow portion 902 (e.g., by the first section 920) and may not be visible through the central flow portion 902 but is shown here for illustrative purposes. In some embodiments, the opening 911 may have an elliptical (e.g., circular) shape. The first section 920 of the flow portion 902 may be configured to at least partially cover the opening 911 in the tissue wall 908 at the first side 901 of the tissue wall 908. In some embodiments, the first section 920 may form an elliptical (e.g., circular) shape around the opening 911. The first section 920 may be secured to the tissue wall 908 (e.g., at the first side 901) through use of one or more anchoring mechanisms 914. In some embodiments, an anchoring mechanism 914 may include a nail, screw, hook, barb, and/or other device configured to penetrate and/or otherwise attach to a surface of the tissue wall 908. Moreover, anchoring mechanisms 914 may pass through the flow portion 902 to pinch the flow portion 902 against the tissue wall 908. While four anchoring mechanisms 914 are shown anchoring the first section 920 of the flow portion 902 to the first side 901 of the tissue wall, any number of anchoring mechanisms 914 may be used. Additional anchoring mechanisms 914 may be used to anchor the flow portion 902 (e.g., the third section 924) to the second side 903 of the tissue wall 908.

At least a portion of the second section 922 may be configured for placement within the opening in the tissue wall 908. The second section 922 may have a generally cylindrical/tubular shape and/or may be configured to be shaped to a generally cylindrical/tubular shape in which the size and/or shape of the second section 922 approximates a size and/or shape of the opening in the tissue wall 908. The second section 922 may be configured to establish a barrier to the inner surface of the opening 911 in the tissue wall 908 to prevent in-growth of tissue after creation of the opening 911. In some embodiments, the second section 922 may be configured to press against the inner surface of the opening 911. Each of the first section, second section, and/or third section 924 may be a separate portion of cloth and/or may form a continuous piece of cloth.

At delivery, the flow portion 902 of the cloth shunt implant 900 may have a longitudinal length and/or may be configured to be stretched to a longitudinal length that is approximately equal to a depth of the opening and/or a thickness of the tissue wall 908. Accordingly, a first end of the flow portion 902 (e.g., the first section 920) may be approximately in-line along a longitudinal axis with the first side 901 of the tissue wall 908 and/or a second end of the flow portion 902 (e.g., the third section 924) may be approximately in-line along the longitudinal axis with the second side 903 of the tissue wall 908. The flow portion 902 may be configured to stretch as a thickness of the tissue wall 908 increases such that the first section 920 remains generally in-line with and/or anchored to the first side 901 of the tissue wall 908 and/or the third section 924 remains generally in-line with and/or anchored to the second side 903 of the tissue wall 908.

The cloth shunt implant 900 may be in a compacted and/or otherwise expandable form at delivery. For example, the cloth shunt implant 900 may be rolled, twisted, relaxed, and/or otherwise compacted to fit into a catheter and/or to allow the cloth shunt implant 900 to be stretched to fit the opening 911 in the tissue wall 908. After delivery of the cloth shunt implant 900, as the tissue wall 908 expands (e.g., longitudinally), the flow portion 902 (e.g., the second section 922) may stretch to create a greater longitudinal length of the cloth shunt implant 900. In some embodiments, the flow portion 902 may have an at least partially elastic structure and/or may resist stretching until a sufficient force is applied (e.g., expansion of the tissue wall 908).

### Delivery Processes

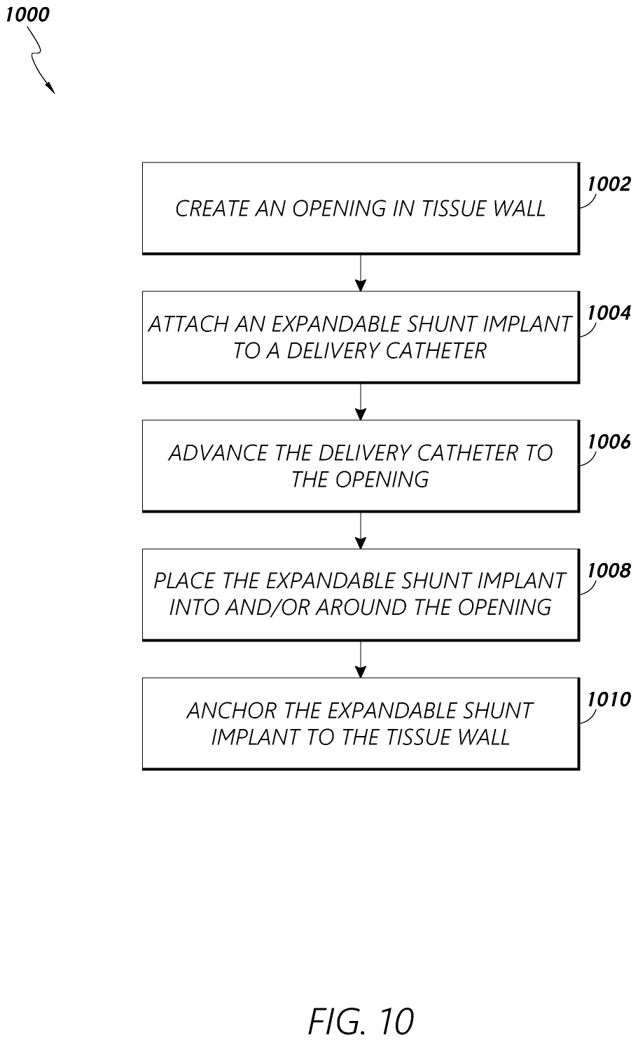

FIG. 10 is a flow diagram of an example of a process 1000 for delivering and/or anchoring an expandable shunt to a body of a person in accordance with some embodiments. In block 1002, the process 1000 involves creating an opening in a tissue wall. As described herein, the opening may be created through use of one or more of a guidewire, puncture catheter, introducer sheath, puncture sheath, and/or puncture expander. The opening may create a blood flow path between two anatomical chambers (e.g., the left atrium and the coronary sinus).

In block 1004, the process 1000 involves attaching an expandable shunt to a delivery catheter. The expandable shunt may be situated within a lumen of the delivery catheter and/or may be in a collapsed state during delivery. In block 1006, the process 1000 involves advancing the delivery catheter to and/or near the opening.

In block 1008, the process 1000 involves placing the expandable shunt into and/or around the opening. For example, the shunt may comprise a flow portion configured to be situated within the opening and/or one or more anchoring mechanisms configured to anchor the flow portion to portions of the tissue wall outside the opening. In block 1010, the process 1000 involves anchoring the expandable shunt to the tissue wall.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

Delivery systems as described herein may be used to position catheter tips and/or catheters to various areas of a human heart. For example, a catheter tip and/or catheter may be configured to pass from the right atrium into the coronary sinus. However, it will be understood that the description can refer or generally apply to positioning of catheter tips and/or catheters from a first body chamber or lumen into a second body chamber or lumen, where the catheter tips and/or catheters may be bent when positioned from the first body chamber or lumen into the second body chamber or lumen. A body chamber or lumen can refer to any one of a number of fluid channels, blood vessels, and/or organ chambers (e.g., heart chambers). Additionally, reference herein to “catheters,” “tubes,” “sheaths,” “steerable sheaths,” and/or “steerable catheters” can refer or apply generally to any type of elongate tubular delivery device comprising an inner lumen configured to slidably receive instrumentation, such as for positioning within an atrium or coronary sinus, including for example delivery catheters and/or cannulas. It will be understood that other types of medical implant devices and/or procedures can be delivered to the coronary sinus using a delivery system as described herein, including for example ablation procedures, drug delivery and/or placement of coronary sinus leads.

What is claimed is:

1. A shunt comprising a central flow portion configured to:
fit at least partially within an opening in a tissue wall, wherein the tissue wall is situated between a first anatomical chamber and a second anatomical chamber, and the opening represents a blood flow path between the first anatomical chamber to the second anatomical chamber;
maintain the blood flow path from the first anatomical chamber to the second anatomical chamber;
prevent in-growth of tissue within the opening; and
expand in response to expansion of the tissue wall.

2. The shunt of claim 1, further comprising one or more anchoring arms extending from the central flow portion, the one or more anchoring arms configured to anchor to the tissue wall.

3. The shunt of claim 1, wherein the central flow portion comprises a network of one or more lines, and each of the one or more lines is configured to interweave with itself or at least one other line of the one or more lines.

4. The shunt of claim 1, wherein the central flow portion comprises a network of interlocking circular wires, and each wire of the network of interlocking circular wires is configured to interlock with at least one other wire of the network of interlocking circular wires.

5. The shunt of claim 1, wherein the central flow portion comprises a coiled line.

6. The shunt of claim 5, wherein the central flow portion has a fixed diameter approximately equal to a diameter of the opening.

7. The shunt of claim 5, wherein:
a first portion of the central flow portion is configured to be situated within the opening;
a second portion of the central flow portion is configured to extend into the first anatomical chamber;
the first portion has a first diameter and the second portion has a second diameter; and
the second diameter is greater than the first diameter.

8. The shunt of claim 1, wherein the central flow portion comprises one or more rings, each of the one or more rings having an elliptical shape to approximate a shape of the opening.

9. The shunt of claim 8, wherein:
each of the one or more rings is composed of a shape-memory material;
each of the one or more rings is configured to naturally assume a first diameter;
each of the one or more rings is configured to be compressed to a second diameter that is lesser than the first diameter to fit into the opening; and
each of the one or more rings is configured to press against the tissue wall to hold itself in place.

10. The shunt of claim 8, wherein each of the one or more rings comprises an anchoring mechanism configured to anchor to the tissue wall.

11. The shunt of claim 1, wherein:
the central flow portion comprises two or more telescoping members;
a first telescoping member of the two or more telescoping members has a first diameter;
a second telescoping member of the two or more telescoping members has a second diameter that is lesser than the first diameter; and
the second telescoping member is configured to fit at least partially within a central opening of the first telescoping member.

12. The shunt of claim 11, wherein the second telescoping member is configured to move with respect to the first telescoping member to adjust an amount of overlap between the first telescoping member and the second telescoping member.

13. The shunt of claim 12, wherein the second telescoping member is configured to decrease the amount of overlap between the first telescoping member and the second telescoping member in response to expansion of the tissue wall.

14. The shunt of claim 13, wherein the first telescoping member and the second telescoping member comprise one or more connection mechanisms configured to allow one-way movement of the second telescoping member.

15. The shunt of claim 1, wherein the central flow portion comprises a sheet of cloth configured to extend from the first anatomical chamber to the second anatomical chamber, and stretch in response to expansion of the tissue wall.

16. The shunt of claim 15, further comprising one or more anchoring mechanisms configured to anchor the sheet of cloth to a first side of the tissue wall.

17. The shunt of claim 16, wherein the sheet of cloth forms a sac, is configured to at least partially cover the opening, and has one or more holes to allow blood flow through the sheet of cloth.

18. A method comprising:
creating an opening in a tissue wall, wherein:
the tissue wall is situated between a first anatomical chamber and a second anatomical chamber; and
the opening represents a blood flow path between the first anatomical chamber to the second anatomical chamber; and
placing a shunt at the opening, the shunt comprising a central flow portion configured to:
fit at least partially within the opening in the tissue wall;
maintain the blood flow path from the first anatomical chamber to the second anatomical chamber;
prevent in-growth of tissue within the opening; and
expand in response to expansion of the tissue wall.

19. The method of claim 18, wherein:
the central flow portion comprises two or more telescoping members;
a first telescoping member of the two or more telescoping members has a first diameter;
a second telescoping member of the two or more telescoping members has a second diameter that is lesser than the first diameter; and
the second telescoping member is configured to fit at least partially within a central opening of the first telescoping member.

20. The method of claim 18, wherein the central flow portion comprises a sheet of cloth configured to extend from the first anatomical chamber to the second anatomical chamber, and stretch in response to expansion of the tissue wall.
